(19) United States
(12) Patent Application Publication
(10) Pub. No.: US 2014/0343459 A1
(43) Pub. Date: Nov. 20, 2014

(54) SPASTICITY MEASUREMENT DEVICE

(71) Applicants: Naoichi Chino, Hachiohji-shi (JP); Yoshihiro Muraoka, Yokohama-shi (JP); Hiroki Ishihama, Tokyo (JP); Shoji Okumura, Nagakute-shi (JP)

(72) Inventors: Naoichi Chino, Hachiohji-shi (JP); Yoshihiro Muraoka, Yokohama-shi (JP); Hiroki Ishihama, Tokyo (JP); Shoji Okumura, Nagakute-shi (JP)

(73) Assignee: TOMEI BRACE CO., LTD., Seto-shi, Aichi (JP)

(21) Appl. No.: 14/363,576

(22) PCT Filed: Dec. 5, 2012
(86) PCT No.: PCT/JP2012/081574

§ 371 Date: Jun. 6, 2014

(30) Foreign Application Priority Data
Dec. 9, 2011 (JP) 2011-270065

(51) Int. Cl. A61B 5/11; A61B 5/22; A61B 5/00

(52) U.S. Cl. CPC A61B 5/1107; A61B 5/6829; A61B 5/1124; A61B 5/4884; A61B 5/4595; A61B 5/224
USPC 600/592

(57) ABSTRACT

A spasticity measurement apparatus includes: a leg mounting fixture including a lower leg mounting portion mounted on a lower leg of a subject for fixation, and a foot receiving portion coupled to a lower end portion of the lower leg mounting portion and having a heel receiving portion and a toe receiving portion for respectively receiving a heel and a toe of a foot leading to the lower leg, the toe receiving portion being disposed relatively rotatably around the heel receiving portion in an approaching rotation direction bringing the toe receiving portion closer to the lower leg mounting portion; a rotation inhibition apparatus inhibiting relative rotation when the foot receiving portion rotates relative to the lower leg mounting portion in the approaching direction; and a muscular contraction force measurement apparatus measuring a muscular contraction force acting in a rotation direction.

### TECHNICAL FIELD

The present invention relates to a measurement apparatus for easily evaluating spasticity or particularly spasticity of a lower limb.

### BACKGROUND ART

Spasticity is a side effect associated with paralysis, means muscle stiffness, uncontrollable leg motion, etc., and includes symptoms such as an increase in muscle tone, sudden muscular contraction, deep tendon hyperreflexia, muscular spasm, scissors-like legs, and fixation of a joint. Treatment of spasticity generally includes motion exercise as well as drug treatment using drugs to remove neurological/muscle disorders.

Such treatment of spasticity requires comprehension of current symptoms of a patient and constant comprehension of changes in symptom during treatment in terms of providing proper treatment. Although spasticity has hitherto been generally evaluated by using the following five-grade Ashworth Scale, this evaluation criterion relies on senses of a physician etc., and inevitably becomes subjective and, therefore, an objective and quantitative evaluation method has been desired. Particularly, drugs removing or alleviating muscle tone have recently been developed and a quantitative evaluation method must be established for evaluating the drugs.

### (Ashworth Scale)

0: normal muscle tone;
1: slight increase in muscle tone causing a catch when limb is moved;
2: more increase in muscle tone than grade 1 allowing easy movement of limb;
3: considerable increase in muscle tone making passive movement of limb difficult; and
4: rigid limb unable to be flexed or extended.

### PRIOR ART DOCUMENTS

### Patent Documents

Patent Document 1: Japanese Patent No. 2586910
Patent Document 2: Japanese Laid-Open Patent Publication No. 2003-319920

### Non-Patent Literature

### Non-Patent Literature 1: “16. Comparison Examination of Measurement of Degree of Lower Limb Spasticity in Hemiplegia” (Japanese Journal of Rehabilitation Medicine, p. 294, vol. 24, no. 5, September 1987)

### SUMMARY OF THE INVENTION

### Problem to Be Solved by the Invention

A foot joint spasticity degree measurement apparatus (see Patent Document 1) and a joint motor function evaluation apparatus (see Patent Document 2) are proposed as apparatuses quantitatively evaluating spasticity.

The former is made up of a pedestal having an L-shaped bottom plate, a side plate, and a lower leg receiving portion; a sole frame and a movable frame disposed rotatably and coaxially on the pedestal; an operation lever operating rotation of the movable frame; a load detector detecting magnitude of a load when the sole frame is rotated by the movable frame in accordance with the rotation operation of the operation lever; an angel detector detecting a rotation angle of the sole frame; and a display/record apparatus displaying/recording the detected load and rotation angle. According to such a spasticity degree measurement apparatus, it is considered that when an experimenter rotates a foot joint of a subject with the movable frame against a rotation resistance force, a stiffness strength and a spasticity degree can continuously be measured at each rotation angle of the joint over the entire movable range of the foot joint of the subject from corresponding rotation angel and rotation resistance force.

The latter is made up of an acceleration signal input apparatus and an evaluation value calculation apparatus. The acceleration signal input apparatus consists of an acceleration sensor portion lifting and freely dropping a predetermined portion of a human body, or giving an electric signal to cause free oscillation of the predetermined portion of a human body, to measure joint angular acceleration of the predetermined portion of a human body, and an acceleration signal amplifying portion amplifying a signal voltage thereof. The acceleration sensor portion consists of two acceleration meters mounted on a lower leg etc. at a constant interval. The evaluation value calculation apparatus consists of an acceleration data capturing portion capturing an amplified joint angular acceleration; an acceleration data calculation processing portion calculating actually measured joint angular acceleration based on the captured joint angular acceleration data; a joint motion simulator portion calculating angular acceleration of joint motion at normal time in a simulated manner by utilizing the actually measured joint acceleration; an evaluation value calculating portion calculating an evaluation value of a joint motor function from the actually measured joint angular acceleration and the angular acceleration of joint motion at normal time calculated in a simulated manner; and an evaluation value output portion outputting information calculated by the evaluation value calculating portion. According to such a joint motor function evaluation apparatus, it is considered that the operation for measurement can be simplified as compared to the spasticity degree measurement apparatus described in Patent Document 1 since an evaluation value of the joint motor function is calculated and output simply by lifting and freely dropping etc. of a lower leg portion of a subject.

However, the apparatus described in Patent Document 1 is an apparatus measuring a rotation resistance force of a foot joint. Although the apparatus described in Patent Document 2 is an apparatus inducing an attenuating motion like a pendulum motion of a foot joint etc. to measure angular acceleration causing the attenuating motion, i.e., acceleration in flexion/extension direction, the acceleration is generated by the same rotation resistance force of a joint as Patent Document 1. Therefore, the both apparatuses only measure joint viscoelasticity and do not measure a spasticity degree. Thus, the apparatuses cannot be used for quantitative comprehension of a symptom of a patient in treatment of spasticity or for evaluation of a new drug and are not used in a clinical manner.

On the other hand, although attempts of quantitative comprehension have been made in medical sites (see, e.g., Non-Patent Literature 1), the attempts remain in the evaluation stage and are not yet in practical use since a large-scale apparatus is required due to usage of muscle measurement/training apparatuses or a sense of anxiety of a subject is easily induced as described in Patent Document 1.

The present invention was conceived in view of the situations and it is therefore an object of the present invention to provide a spasticity measurement apparatus capable of easily measuring a spasticity degree particularly for spasticity of a lower limb.

### Means for solving the Problem

To achieve the object, the first aspect of the invention provides a spasticity measurement apparatus comprising: (a) a leg mounting fixture including a lower leg mounting portion mounted on a lower leg of a subject for fixation, and a foot receiving portion coupled to a lower end portion of the lower leg mounting portion and having a heel receiving portion and a toe receiving portion for respectively receiving a heel and a toe of a foot leading to the lower leg, the toe receiving portion being disposed relatively rotatably around the heel receiving portion in an approaching rotation direction bringing the toe receiving portion closer to the lower leg mounting portion; (b) a rotation inhibition apparatus inhibiting relative rotation when the foot receiving portion rotates relative to the lower leg mounting portion in the approaching direction; and (c) a muscular contraction force measurement apparatus measuring a muscular contraction force acting in a rotation direction while the foot receiving portion is inhibited from rotating by the rotation inhibition apparatus.

The second aspect of the invention provides a spasticity measurement apparatus measuring a spasticity degree by inducing spasticity of a lower limb and by measuring a contraction force of muscular repeated contraction generated accordingly, comprising: (a) a leg mounting fixture including a lower leg mounting portion mounted on a lower leg of a subject for fixation, and a foot receiving portion coupled to a lower end portion of the lower leg mounting portion and having a heel receiving portion and a toe receiving portion for respectively receiving a heel and a toe of a foot leading to the lower leg, the toe receiving portion being disposed relatively rotatably around the heel receiving portion in an approaching rotation direction bringing the toe receiving portion closer to the lower leg mounting portion; and (b) a leg mounting fixture support stand including a heel support portion and a toe support portion supporting the heel receiving portion and the toe receiving portion, respectively, at predetermined heights, and a support release apparatus for instantaneously releasing support by the heel support portion.

### Effects of the Invention

According to the first aspect of the invention, the spasticity measurement apparatus includes the leg mounting fixture having the lower leg mounting portion and the foot receiving portion coupled relatively rotatably to each other and, when the lower leg mounting portion and the foot receiving portion are relatively rotated, the rotation is inhibited by the rotation inhibition apparatus, and the muscular contraction force acting in the rotation direction is measured in this state by the muscular contraction force measurement apparatus. Therefore, since a subject having the spasticity induced by the relative rotation of the foot receiving portion and the lower leg mounting portion is caused to generate rhythmic repeated contraction with lengths of lower leg flexor muscles fixed in a stretched state, a spasticity degree can easily be measured by measuring the muscular contraction force acting in the rotation direction of the foot receiving portion by the muscular contraction force measurement apparatus. The quantitative measurement of the spasticity degree can also easily be performed.

According to the second aspect of the invention, in the spasticity measurement apparatus, the leg mounting fixture having the lower leg mounting portion and the foot receiving portion coupled relatively rotatably to each other is supported by the leg mounting fixture support stand receiving the heel receiving portion and the toe receiving portion of the foot receiving portion and, therefore, when the leg mounting fixture is mounted on the lower limb of the subject, the lower limb is supported by the leg mounting fixture support stand. Since the leg mounting fixture support stand includes the support release apparatus for instantaneously releasing support by the heel support portion receiving the heel receiving portion, when support by the heel support portion is released by the support release apparatus, the heel receiving portion falls due to the weight of the leg of the subject and the toe receiving portion is kept supported by the toe support portion and, therefore, the lower limb and the foot of the subject are relatively rotated in the stretching direction of the lower leg flexor muscles i.e. in the dorsiflexion direction, thereby causing the lower leg mounting portion and the foot receiving portion mounted thereon to relatively rotate. Because the spasticity is induced by instantaneously stretching the lower leg flexor muscles in this way, the spasticity degree can quantitatively be measured by measuring contraction force of muscular repeated contraction generated in this case. In other words, according to the present invention, since the support of the heel receiving portion can instantaneously be released in this configuration, the spasticity degree can easily be evaluated with excellent reproducibility.

The first aspect of the invention preferably comprises a foot receiving portion rotation apparatus for rotating the foot receiving portion relative to the lower leg mounting portion in the approaching rotation direction. Although a mechanism relatively rotating the foot receiving portion and the lower leg mounting portion can be disposed outside or another person can rotate a leg of a subject with an appropriate method instead of disposing such a mechanism, the configuration including the foot receiving portion rotating apparatus as described above is preferable.

The above-described foot receiving portion rotation apparatus is a leg mounting fixture support stand including a heel support portion and a toe support portion supporting the heel receiving portion and the toe receiving portion, respectively, at predetermined heights, and a support release apparatus for instantaneously releasing support by the heel support portion. Therefore, the leg mounting fixture support stand included in the spasticity measurement apparatus of the second aspect of the invention can be disposed as the foot receiving portion rotating apparatus.

Preferably, the heel support portion includes a first rotation arm attached at one end portion thereof to a base rotatably around an axis parallel to a rotation axis of the foot receiving portion, and a second rotation arm attached to an other end portion of the first rotation arm rotatably around an axis parallel to the axis. The second rotation arm supports the heel receiving portion. As a result, when the first rotation arm and the second rotation arm included in the heel supporting portion are relatively rotated, a distance can be made shorter between one end portion of the first rotation arm attached to a base and the supporting portion of the heel receiving portion of the second rotation arm, so as to easily instantaneously release the support by the heel supporting portion.

Preferably, the second rotation arm forms a longitudinal shape slightly bent at a center portion to which the other end portion of the first rotation arm is attached, and at a rotation position bringing one end portion of the second rotation arm into contact with the first rotation arm, the second rotation arm supports the heel receiving portion with the other end portion. As a result, the support of the heel receiving portion can easily be released by pulling one end portion of the second rotation arm.

Preferably, the spasticity measurement apparatus comprises a first rotation arm support member supporting the first rotation arm rotating in a first direction such that the first rotation arm is slightly tilted in the rotation direction thereof from a vertical direction, and a second rotation arm support member supporting the second rotation arm rotating in a second direction opposite to the first direction such that the second rotation arm is slightly tilted in the rotation direction thereof from the vertical direction. As a result, since the first rotation arm and the second rotation arm are somewhat bent as a whole to receive the heel receiving portion, when force is applied to a coupling portion thereof to cause relative rotation, the bending state can be changed to the opposite direction so as to easily instantaneously release the support by the heel supporting portion.

Preferably, the spasticity measurement apparatus comprises a rotation inhibition apparatus inhibiting relative rotation when the foot receiving portion is caused to rotate in the approaching rotation direction relative to the lower leg mounting portion as the support is released, and a muscular contraction force measurement apparatus measuring a muscular contraction force acting in a rotation direction of the foot receiving portion while the foot receiving portion is inhibited from rotating by the rotation inhibition apparatus. As a result, when the foot receiving portion is rotated relative to the lower leg mounting portion by releasing the support, the foot receiving portion is fixed at a rotated position by the rotation inhabitation apparatus. Therefore, since a subject with spasticity induced is caused to generate rhythmic repeated contraction while lengths of lower leg flexor muscles are kept fixed in a stretched state when the heel falls, a spasticity degree can easily quantitatively be measured by measuring the muscular contraction force acting in the rotation direction of the foot receiving portion by the muscular contraction force measurement apparatus.

A spasticity degree indicates a level of facilitation of monosynaptic reflex realizing muscular contraction when the lower leg flexor muscles are instantaneously stretched, and a level of subsequent clonus (i.e., rhythmic repeated contraction). For example, in the case of a foot joint, spasticity is a level of facilitation of monosynaptic reflex ascending Ia nerve fibers from muscular spindles, monosynaptically exciting anterior horn cells in the spinal cord, descending motor nerves, and realizing muscular contraction when the lower leg flexor muscles (i.e., soleus muscle and gastrocnemius muscle) are instantaneously stretched, and a level of subsequent clonus (rhythmic repeated contraction). Although differing depending on nerve conduction velocity and leg length, a time required from the stretch to the start of contraction of muscle is about 40 (ms), for example. A level of induced spasticity also depends on a stretching speed of muscle. Therefore, to measure a spasticity degree, the following three points are necessary. As described above, since the muscle length is fixed by fixing the rotation when the heel falls, a spasticity degree can easily be measured.

(1) Muscle is instantaneously stretched with excellent reproducibility.

(2) Muscle length is fixed until reflex occurs (muscular contraction starts).

(3) Muscular contraction force is measured.

Preferably, the rotation inhibition apparatus includes a rotating member disposed coaxially to a rotation axis of the foot receiving portion to rotate integrally with the foot receiving portion, and a rotation stop apparatus disposed on the lower leg mounting portion so as to stop rotation of the rotating member.

Preferably, the rotating member is an annular cam having a cutout in a portion in a circumferential direction, and the rotation stop apparatus has an engagement projecting portion pressed by a biasing member against the cam in a circumferentially immovable state from an outer circumferential side and fitted into the cutout to inhibit rotation of the cam. As a result, the rotation of the foot receiving portion can certainly be inhibited with a simple configuration by the cam and the engagement projecting portion fitted into the cutout. The biasing member is, for example, an elastic member such as a compression coil spring, a band-like spring, and rubber or an actuator such as a hydraulic cylinder and an air cylinder.

Preferably, the muscular contraction force measurement apparatus detects strain of the lower leg mounting portion with a strain gauge to measure a muscular contraction force. As a result, since the muscular contraction force transmitted to the lower limb mounting portion is detected by the strain gauge, the muscular contraction force can easily be measured by processing an output signal thereof. The muscular contraction force may be measured by measuring rotation torque acting on a rotation shaft relatively rotatably coupling the lower limb mounting portion and the foot receiving portion with a torque sensor etc., instead of, or in addition to, the strain gauge.

Preferably, the muscular contraction force measurement apparatus includes an angle detection apparatus for detecting a relative rotation angle between the lower leg mounting portion and the foot receiving portion. As a result, since a change in muscular contraction force can be acquired from a relationship with an angle of the foot relative to the lower leg portion, the spasticity degree can more easily be evaluated.

Preferably, the rotation inhibition apparatus inhibits rotation of the foot receiving portion when the heel receiving portion reaches a lowest point after the support release apparatus is actuated. As a result, since the rotation of the foot receiving portion is immediately inhibited when the heel receiving portion falls to the lowest point by releasing the support, the rotation of the foot receiving portion is inhibited until muscular contraction occurs due to monosynaptic reflex and, therefore, the muscular contraction force from facilitation of muscular contraction and subsequent rhythmic repeated contraction can certainly be measured and the spasticity degree can more certainly be measured in a quantitative manner.

The present invention is preferably configured as a spasticity measurement system including the spasticity measurement apparatus and a force change acquisition means acquiring temporal changes in respective forces in dorsiflexion and plantar flexion directions of a foot joint based on a change in the returning force of the spasticity measurement apparatus. As a result, data required for spasticity evaluation can be acquired based on a change in muscular contraction measured by the spasticity measurement apparatus.

### BRIEF DESCRIPTION OF THE DRAWINGS

FIG. 1 is a diagram of an overall configuration of a spasticity measurement apparatus of an embodiment of the present invention.

FIG. 2 is a diagram for explaining a contraction force detection method in the spasticity measurement apparatus of FIG. 1.

FIG. 3 is a diagram for explaining a rotation inhibition apparatus included in the spasticity measurement apparatus of FIG. 1.

FIG. 4 is a diagram for showing an operational state of the rotation inhibition apparatus of FIG. 3.

FIG. 5 is a diagram for showing an operational state of inducing spasticity by the spasticity measurement apparatus of FIG. 1.

FIG. 6 is a diagram for depicting an example of the measurement result by the spasticity measurement apparatus of FIG. 1.

FIG. 7 is a diagram for explaining another example of configuration of the support release apparatus.

### MODE FOR CARRYING OUT THE INVENTION

An embodiment of the present invention will now be described in detail with reference to the drawings. In the following embodiment, the figures are simplified or deformed as needed and portions are not necessarily precisely depicted in terms of dimension ratio, shape, etc.

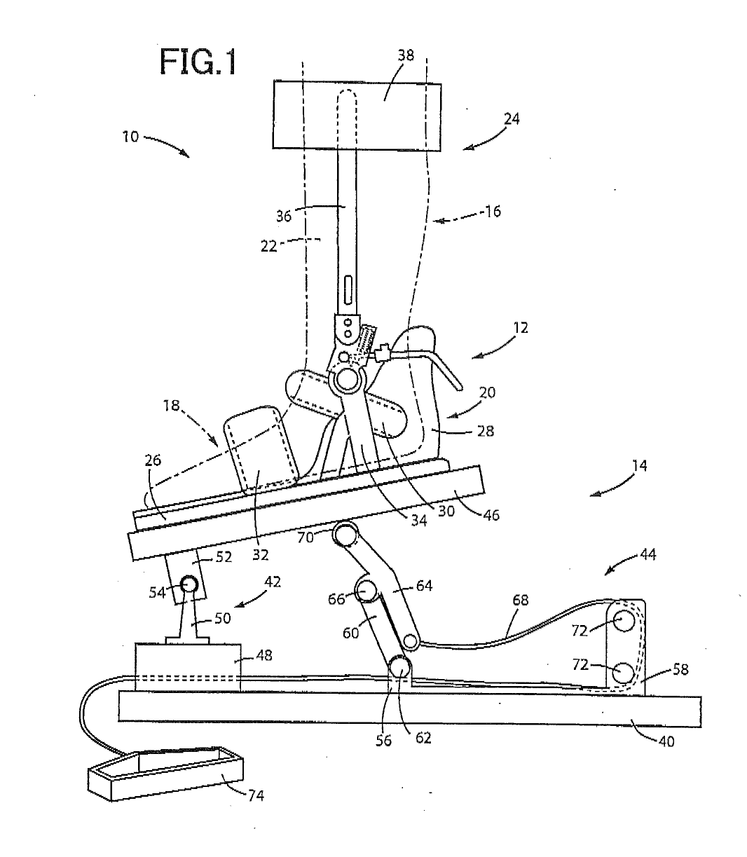

FIG. 1 is a diagram of an overall configuration of a spasticity measurement apparatus 10 of an embodiment of the present invention. In FIG. 1, the spasticity measurement apparatus 10 includes a leg mounting fixture 12 and a leg mounting fixture support stand 14.

The leg mounting fixture 12 has a foot receiving portion 20 retaining a foot 18 of a lower limb 16 of a subject indicated by a dashed-dotted line of FIG. 1, and a lower leg mounting portion 24 for mounting on a lower leg portion 22. The foot receiving portion 20 includes a plate-like member 26 made of rubber etc. and has a heel receiving portion 28 for receiving a heel and belts 30, 32 for fixing the foot 18 to the side surfaces and the front side (the left side of FIG. 1) of the heel receiving portion 28 attached onto the plate-like member 26, and a toe portion of the foot 18 is received by a left portion of the plate-like member 26 of FIG. 1 opposite to the heel receiving portion 28.

A pair of fixation-side support members 34 (depicted only on one side located on the near side; the same applies to the following description) is projected substantially upward from the both lateral sides of the foot receiving portion 20. The lower leg mounting portion 24 includes a pair of rotation-side support members 36 attached to respective upper end portions of the pair of the fixation-side support members 34 rotatably around an axis perpendicular to the plane of FIG. 1, i.e., in the substantially horizontal direction, and a lower limb upper portion fixture 38 attached to upper end portions of the rotation-side support members 36.

The leg mounting fixture support stand 14 includes a flat plate-shaped base 40, a pair of toe support stands 42 and a heel support stand 44 fixed to the base 40, and a foot receiving portion support plate 46 supported by the toe support stands 42 and the heel support stand 44 at a position above the base 40. The plate-like member 26, the fixation-side support members 34, etc. are fixed onto the foot receiving portion support plate 46. The pair of the toe support stands 42 includes respective bearings 50 on pedestals 48, and a rotation shaft 54 penetrates a pair of projecting pieces 52 projected from a lower surface of the foot receiving portion support plate 46 so that the foot receiving portion support plate 46 is supported rotatably around the rotation shaft 54.

The heel support stand 44 has a rotation arm support portion 56 and a pulley support portion 58 integrally disposed, and a first rotation arm 60 is attached at one end portion to the rotation arm support portion 56 rotatably around a rotation shaft 62 and is somewhat tilted to the left side, i.e., toward the toe support stands 42. To the other end portion of the first rotation arm 60, a longitudinal second rotation arm 64 bent at a center portion is attached rotatably around a rotation shaft 66 at a projecting portion disposed on a valley side of a bending portion at the center portion. One end portion of the second rotation arm 64 located on the lower side in FIG. 1 is brought into contact with the first rotation arm 60 and is tilted at the same angle as the first rotation arm 60 as a whole and a wire 68 is attached to the one end portion for pulling the one end portion to the right side in FIG. 1. The other end portion of the second rotation arm 64 is located above the first rotation arm 60 to support the foot receiving portion support plate 46 with a roller 70 disposed at the other end portion. Therefore, the second rotation arm 64 is not attached to the foot receiving portion support plate 46 and only receives the foot receiving portion support plate 46 from a lower side.

The wire 68 is pulled via two pulleys 72, 72 disposed on the pulley support portion 58 to the left side in FIG. 1 and is connected at the end to a grip 74 used for pulling the wire 68.

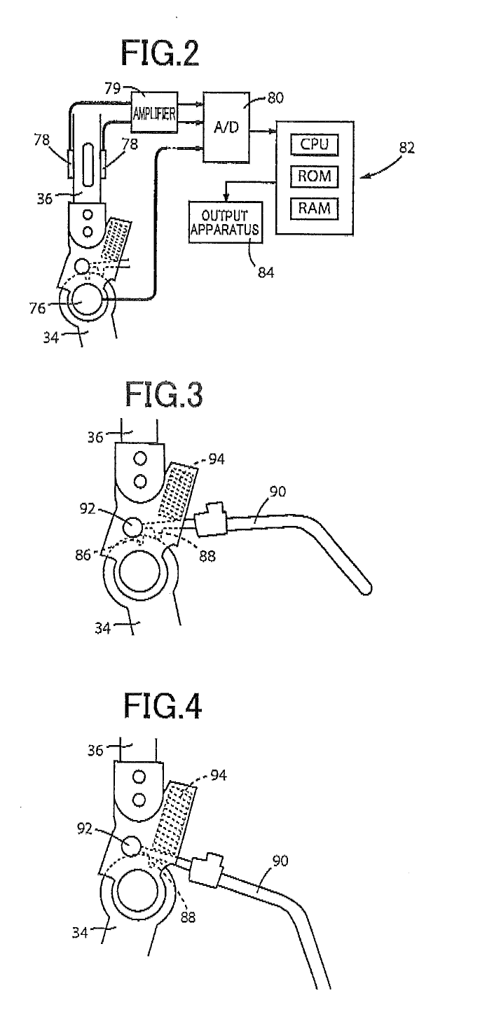

FIG. 2 is an enlarged detailed view of a vicinity of a coupling portion of the fixation-side support member 34 and the rotation-side support member 36. A potentiometer 76 is attached to a rotation shaft thereof such that a rotation angle of the rotation-side support member 36 can be detected as an electric signal. Strain gauges 78 are respectively affixed to a front-side side surface and a rear-side side surface near lower ends of a pair of the rotation-side support members 36 and, if a pulling force or a compressing force acting on the front-side side surface and the rear-side side surface causes strain in the rotation-side support member 36, an electric signal corresponding to the change can be detected from the strain gauges 78.

The electric signal detected from the potentiometer 76 and the electric signal detected by the strain gauges 78 and amplified by an amplifier 79 are sent via an A/D converter 80 to a data processing apparatus 82. The data processing apparatus 82 is a computer including a CPU, a ROM, a RAM, etc., and the CPU continuously executes arithmetic processing of a strain amount, a rotation angle, etc. in accordance with a program stored in advance in the ROM or the RAM, while utilizing a temporary storage function of the RAM, to sequentially store temporal changes thereof or output the temporal changes from an output apparatus 84 such as a printer and a monitor.

FIG. 3 is a further enlarged view of the coupling portion. An upper end of the fixation-side support member 34 forms an arc shape and includes a shallow engagement concave portion 86 at a portion in circumferential direction. On the other hand, to a lower end portion of the rotation-side support member 36, an engagement lever 90 having a projection 88 projected toward an upper end outer circumferential surface of the fixation-side support member 34 is attached rotatably around a rotation shaft 92, and a compression coil spring 94 biasing the engagement lever 90 toward the upper end portion of the fixation-side support member 34 is disposed in the vicinity thereof. Therefore, the engagement lever 90 has the projection 88 always pressed toward the upper end outer circumferential surface of the fixation-side support member 34.

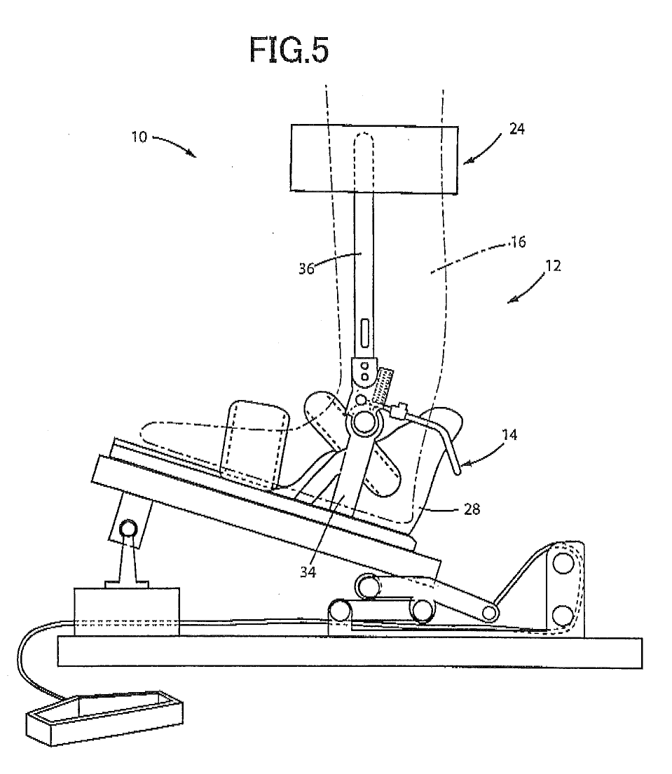

FIG. 4 depicts a state of the fixation-side support member 34 and the rotation-side support member 36 slightly relatively rotated to a relative rotation position changed from the state depicted in FIG. 3. When the relative rotation position is changed in this way and the projection 88 is displaced to a position of the engagement concave portion 86, the engagement lever 90 biased by the compression coil spring 94 is rotated around the rotation shaft 92 toward the fixation-side support member 34 and, therefore, the projection 88 fits into the engagement concave portion 86. In this fitted state, the engagement lever 90 is being pressed by the compression coil spring 94 against the fixation-side support member 34 and, therefore, the relative rotation is inhibited between the fixation-side support member 34 and the rotation-side support member 36. In other words, when the fixation-side support member 34 and the rotation-side support member 36 are relatively rotated by a predefined angle, the relative rotation position thereof is fixed. In this embodiment, the projection 88, the engagement concave portion 86, the compression coil spring 94, etc., make up a rotation inhibition apparatus.

When a spasticity degree of the lower limb 16 of the subject is measured by using the spasticity measurement apparatus 10 configured in this way, the foot 18 of the lower limb 16 is placed on the foot receiving portion 20 as indicated by the dashed-dotted line while the foot receiving portion support plate 46 is supported by the second rotation arm 64 as depicted in FIG. 1 and the leg mounting fixture 12 is accordingly supported by the leg mounting fixture support stand 14. The foot 18 is fixed by the belts 30, 32 and the lower limb upper portion fixture 38 is attached to an upper end portion of the lower limb 16, for example, a portion slightly lower than the knee. Although the lower limb upper portion fixture 38 must not be fastened too tight, the lower limb upper portion fixture 38 is preferably fastened such that a gap is eliminated as far as possible so as to enable accurate torque measurement. In the initial state described above, an angle of the foot 18 relative to the lower leg portion 22 is set to 10 degrees in plantar flexion, for example.

A measurement start operation of the data processing apparatus 82 activated in advance is performed in this state and, thereafter, when the grip 74 is held and the wire 68 is swiftly pulled, one end portion of the second rotation arm 64 is pulled to the right side of FIG. 1 by the wire 68 and is rotated counterclockwise around the rotation shaft 66, and the first rotation arm 60 is rotated via the rotation shaft 66 clockwise around the rotation shaft 62. As a result, since a position of the roller 70 is instantaneously lowered, the foot receiving portion support plate 46 loses support and is rotated around the rotation shaft 54. In this embodiment, the first rotation arm 60, the second rotation arm 64, the wire 68, etc. make up a support release apparatus.

As a result, the heel receiving portion 28 instantaneously falls due to the weight of the lower limb 16 of the subject and the leg mounting fixture 12 is put into the state depicted in FIG. 5. Therefore, since the leg mounting fixture 12 has the lower leg mounting portion 24 (the rotation-side support member 36) rotated counterclockwise relative to the foot receiving portion 20 (fixation-side support member 34), the lower leg flexor muscles are instantaneously stretched and the spasticity is induced in the lower limb 16 of the subject. Even when the lower leg mounting portion 24 is rotated in this way, the rotation-side support members 36 are kept in orientation along the substantially vertical direction. In this case, as depicted in FIGS. 3 and 4, when the rotation-side support member 36 is rotated in a counterclockwise direction of FIGS. 3 and 4, the projection 88 fits into the engagement concave portion 86 to inhibit the rotation and, therefore, at the moment of stretch of the lower leg flexor muscles, the length of the lower limb 16 of the subject is fixed in this state. Positions of the projection 88 and the engagement concave portion 86 are defined such that this fixation is achieved when the dorsiflexion angle reaches five degrees.

Therefore, since the induced spasticity, i.e., facilitation of monosynaptic reflex or subsequent clonus (rhythmic repeated contraction), occurs while lengths of the lower leg flexor muscles are kept fixed, the repeated contraction bends the rotation-side support members 36. Since the bend is detected as electric signals due to strain of the strain gauges 78, when the electric signals are continuously collected and processed by the data processing apparatus 82, a temporal change in muscular contraction force causing the strain is acquired and output from the output apparatus 84. In other words, quantitative data of the spasticity degree can be acquired. In this embodiment, the strain gauges 78, the data processing apparatus 82, etc. make up a muscular contraction force measurement apparatus.

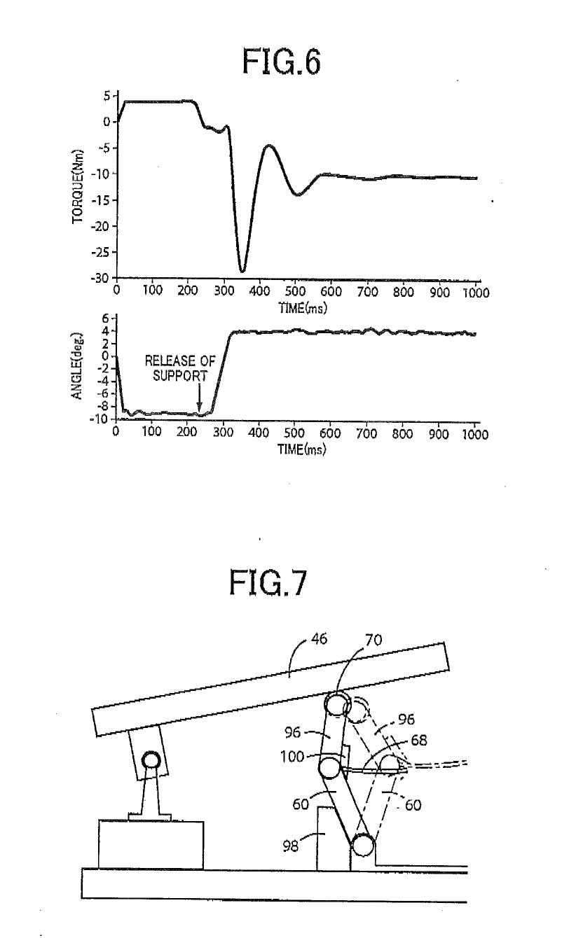

Changes in muscular contraction force measured as described above are depicted in FIG. 6. In FIG. 6, the horizontal axis indicates elapsed time (ms) and the vertical axis of an upper graph indicates a torque value (Nm) of the muscular contraction force acquired from output signals of the strain gauges 78 while the vertical axis of a lower graph indicates a rotation angle of the foot receiving portion 20 acquired from output signal of the potentiometer 76. In the graph of muscular contraction force, a torque in the dorsiflexion direction and a torque in the plantar flexion direction are indicated by a negative value and a positive value, respectively. The rotation angles in the dorsiflexion direction and the plantar flexion direction are indicated by a positive value and a negative value, respectively. When a support by the heel support stand 44 is released near 230 (ms), the heel falls and therefore the rotation angle immediately significantly changes to a positive value. Since the rotation angle of the foot receiving portion 20 is fixed in the rotated state and the spasticity is induced, a torque in the dorsiflexion direction significantly increases immediately after the release of the support; however, because the rhythmic repeated contraction occurs, the torque in the dorsiflexion direction repeatedly increases and decreases, gradually converging to a smaller value. Although the angle of the foot receiving portion 20 is fixed, slight displacements are generated due to the spasticity and, therefore, subtle angle changes continuously occur during the measurement.

In the measurement described above, the torque in the dorsiflexion direction immediately after the release of the support mainly reflects the muscle tone of foot joint plantar flexor muscle and the joint viscoelasticity and a higher peak value is generally acquired from a person having a more flexible foot joint. On the other hand, a higher torque in the plantar flexion direction indicates that stretch reflex is more facilitated and a continuous peak in the plantar flexion direction indicates detection of clonus. Although a total measurement time is five seconds, FIG. 6 depicts only 1000 (ms) before and after the start of measurement.

After the measurement is completed, if the engagement lever 90 is rotated counterclockwise against the biasing force of the compression coil spring 94, fitting between the projection 88 and the engagement concave portion 86 is released and, therefore, restriction of the lower leg flexor muscles is released.

As described above, according to this embodiment, the spasticity measurement apparatus 10 includes the leg mounting fixture 12 having the lower leg mounting portion 24 and the foot receiving portion 20 coupled relatively rotatably to each other and, when the lower leg mounting portion 24 and the foot receiving portion 20 are relatively rotated, the rotation is inhibited by the rotation inhibition apparatus consisting of the projection 88, the engagement concave portion 86, the compression coil spring 94, etc., and the muscular contraction force acting in the rotation direction is measured in this state by the muscular contraction force measurement apparatus made up of the strain gauges 78, the data processing apparatus 82, etc. Therefore, since a subject having the spasticity of the lower limb 16 induced by the relative rotation of the foot receiving portion 20 and the lower leg mounting portion 24 is caused to generate rhythmic repeated contraction with lengths of lower leg flexor muscles fixed in a stretched state, a spasticity degree can easily be measured by measuring the muscular contraction force acting in the rotation direction of the foot receiving portion by the muscular contraction force measurement apparatus. The quantitative measurement of the spasticity degree can also easily be performed.

According to this embodiment, in the spasticity measurement apparatus 10, the leg mounting fixture 12 having the lower leg mounting portion 24 and the foot receiving portion 20 coupled relatively rotatably to each other is supported by the leg mounting fixture support stand 14 receiving the heel receiving portion 28 and a toe receiving portion (a leading end portion of the foot receiving portion 20) of the foot receiving portion 20 and, therefore, when the leg mounting fixture 12 is mounted on the lower limb 16 of the subject, the lower limb 16 is supported by the leg mounting fixture support stand 14. Since the leg mounting fixture support stand 14 is formed such that the support by the heel support stand 44 receiving the heel receiving portion 28 may instantaneously be released, the heel receiving portion 28 falls due to the weight of the leg of the subject and the toe receiving portion is kept supported by the toe support stands 42 and, therefore, the lower leg 16 and the foot 18 of the subject are relatively rotated in the stretching direction of the lower leg flexor muscles, thereby causing the lower leg mounting portion 24 and the foot receiving portion 20 mounted thereon to relatively rotate. Because the spasticity is induced by instantaneously stretching the lower leg flexor muscles in this way, the spasticity degree can quantitatively be measured by measuring contraction force of muscular repeated contraction generated in this case. In other words, since the support of the heel receiving portion 28 can instantaneously be released in this configuration, the spasticity degree can easily be evaluated with excellent reproducibility.

FIG. 7 is a diagram for explaining a main configuration of another embodiment of the present invention. In this embodiment, a second rotation arm 96 having a different shape is included instead of the second rotation arm 64. The second rotation arm 96 forms the longitudinal shape same as the first rotation arm 60 and has one end portion rotatably attached to the first rotation arm 60 and the other end portion supporting the foot receiving portion support plate 46. To the left of the first rotation arm 60, a rotation limiting member 98 is fixed that limits the left rotation thereof, and a rotation limiting plate 100 is attached to a side surface of the second rotation arm 96 to inhibit further rotation to the right from the depicted position.

The wire 68 is attached to a coupling portion between the first rotation arm 60 and the second rotation arm 96. Therefore, when the wire 68 is pulled to the right in this embodiment, the coupling portion is pulled to the right and the first rotation arm 60 is caused to rotate to the right and the second rotation arm 96 is caused to rotate to the left. As a result, since the height of the roller 70 is instantaneously lowered as indicated by a broken line, a right end portion of the foot receiving portion support plate 46 of FIG. 7 falls as is the case with the embodiment and the spasticity can easily and certainly be induced in the same way. In a support release apparatus depicted in FIG. 7, the configuration of the members for limiting the rotation of the first rotation arm 60 and the second rotation arm 96 is an example and an appropriate configuration is employable to the extent that the rotation is limited and that the foot receiving portion support plate 46 can certainly be supported until the support is released.

Although the present invention has been described in detail with reference to the drawings, the present invention can be implemented in other forms and may variously be modified within a range not departing from the spirit thereof.

### NOMENCLATURE OF ELEMENTS

10: spasticity measurement apparatus 12: leg mounting fixture 14: leg mounting fixture support stand 16: lower limb 18: foot 20: foot receiving portion 22: lower leg portion 24: lower leg mounting portion 26: plate-like member 28: heel receiving portion 30, 32: belt 34: fixation-side support members 36: rotation-side support members 38: lower limb upper portion fixture 40: base 42: toe support stands 44: heel support stand 46: foot receiving portion support plate 48: pedestals 50: bearings 52: projecting pieces 54: rotation shaft 56: rotation arm support portion 58: pulley support portion 60: first rotation arm 62: rotation shaft 64: second rotation arm 66: rotation shaft 68: wire 70: roller 72: pulley 74: grip 76: potentiometer 78: strain gauges 80: A/D converter 82: data processing apparatus 84: output apparatus 86: engagement concave portion 88: projection 90: engagement lever 92: rotation shaft 94: compression coil spring 96: second rotation arm 98: rotation limiting member 100: rotation limiting plate

1. A spasticity measurement apparatus comprising:
a leg mounting fixture including a lower leg mounting portion mounted on a lower leg of a subject for fixation, and a foot receiving portion coupled to a lower end portion of the lower leg mounting portion and having a heel receiving portion and a toe receiving portion for respectively receiving a heel and a toe of a foot leading to the lower leg, the toe receiving portion being disposed relatively rotatably around the heel receiving portion in an approaching rotation direction bringing the toe receiving portion closer to the lower leg mounting portion;
a rotation inhibition apparatus inhibiting relative rotation when the foot receiving portion rotates relative to the lower leg mounting portion in the approaching direction; and
a muscular contraction force measurement apparatus measuring a muscular contraction force acting in a rotation direction while the foot receiving portion is inhibited from rotating by the rotation inhibition apparatus.

2. The spasticity measurement apparatus of claim 1, comprising a foot receiving portion rotation apparatus for rotating the foot receiving portion relative to the lower leg mounting portion in the approaching rotation direction.

3. A spasticity measurement apparatus measuring a spasticity degree by inducing spasticity of a lower limb and by measuring a contraction force of muscular repeated contraction generated accordingly, comprising:
a leg mounting fixture including a lower leg mounting portion mounted on a lower leg of a subject for fixation, and a foot receiving portion coupled to a lower end portion of the lower leg mounting portion and having a heel receiving portion and a toe receiving portion for respectively receiving a heel and a toe of a foot leading to the lower leg, the toe receiving portion being disposed relatively rotatably around the heel receiving portion in an approaching rotation direction bringing the toe receiving portion closer to the lower leg mounting portion; and
a leg mounting fixture support stand including a heel support portion and a toe support portion supporting the heel receiving portion and the toe receiving portion, respectively, at predetermined heights, and a support release apparatus for instantaneously releasing support by the heel support portion.

4. The spasticity measurement apparatus of claim 3, wherein the heel support portion includes a first rotation arm attached at one end portion thereof to a base rotatably around a first axis parallel to a rotation axis of the foot receiving portion, and a second rotation arm attached to an other end portion of the first rotation arm rotatably around a second axis parallel to the first axis, and wherein the second rotation arm supports the heel receiving portion.

5. The spasticity measurement apparatus of claim 4, wherein the second rotation arm forms a longitudinal shape slightly bent at a center portion to which the other end portion of the first rotation arm is attached, and wherein at a rotation position bringing one end portion of the second rotation arm into contact with the first rotation arm, the second rotation arm supports the heel receiving portion with the other end portion.

6. The spasticity measurement apparatus of claim 4, comprising a first rotation arm support member supporting the first rotation arm such that the first rotation arm is slightly tilted in a first direction from a vertical direction, and a second rotation arm support member supporting the second rotation arm such that the second rotation arm is slightly tilted in a second direction opposite to the first direction from the vertical direction.

7. The spasticity measurement apparatus of claim 3, comprising
a rotation inhibition apparatus inhibiting relative rotation when the foot receiving portion is caused to rotate in the approaching rotation direction relative to the lower leg mounting portion as the support is released, and
a muscular contraction force measurement apparatus measuring a muscular contraction force acting in a rotation direction of the foot receiving portion while the foot receiving portion is inhibited from rotating by the rotation inhibition apparatus.

8. The spasticity measurement apparatus of claim 7, wherein the rotation inhibition apparatus includes a rotating member disposed coaxially to a rotation axis of the foot receiving portion to rotate integrally with the foot receiving portion, and a rotation stop apparatus disposed on the lower leg mounting portion so as to stop rotation of the rotating member.

9. The spasticity measurement apparatus of claim 8, wherein the rotating member is an annular cam having a cutout in a portion in a circumferential direction, and wherein the rotation stop apparatus has an engagement projecting portion pressed by a biasing member against the cam in a circumferentially immovable state from an outer circumferential side and fitted into the cutout to inhibit rotation of the cam.

10. The spasticity measurement apparatus of claim 7, wherein the muscular contraction force measurement apparatus detects strain of the lower leg mounting portion with a strain gauge to measure a muscular contraction force.

11. The spasticity measurement apparatus of claim 7, wherein the muscular contraction force measurement apparatus includes an angle detection apparatus for detecting a relative rotation angle between the lower leg mounting portion and the foot receiving portion.

12. The spasticity measurement apparatus of claim 7, wherein the rotation inhibition apparatus inhibits rotation of the foot receiving portion when the heel receiving portion reaches a lowest point after the support release apparatus is actuated.
